(19) United States
(12) Patent Application Publication
(10) Pub. No.: US 2022/0054212 A1
(43) Pub. Date: Feb. 24, 2022

(54) Medical Drape with Integrated Toolkit for Rapid Response Care

(71) Applicant: THE REGENTS OF THE UNIVERSITY OF MICHIGAN, Ann Arbor, MI (US)

(72) Inventors: Kevin R. Ward, Superior Township, MI (US); Jeffrey Stephen Plott, Algonac, MI (US)

(21) Appl. No.: 17/406,869

(22) Filed: Aug. 19, 2021

Related U.S. Application Data
(60) Provisional application No. 63/067,648, filed on Aug. 19, 2020.

(51) Int. Cl. A61B 46/23; A61B 50/30

(52) U.S. Cl. CPC A61B 46/23; A61B 50/30

(57) ABSTRACT

Quick-deployable medical drapes with included integrated toolkits are provided that allow the medical drape, once deployed, to simultaneously protect an area of a patient in need of a medical procedure and expose tools of the integrated toolkit for immediate access by medical professionals. The integrated toolkits may be of different types that include medical and/or surgical tools for use medical procedures. The medical drapes are formed of fully or partially sterile materials and formed into packages having a stored state, in which the medical drape and toolkit are protected against contamination, and a deployed state, in which the medical drape is placed on the patient and ready for the medical procedure.

### CROSS-REFERENCE TO RELATED APPLICATION

Priority is claimed to U.S. Provisional Patent Application No. 63/067,648, filed Aug. 19, 2020, the entire disclosure of which is incorporated herein by reference.

### FIELD OF THE DISCLOSURE

The present disclosure relates generally to medical drapes and, more particularly, to medical drapes with integrated toolkits.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Medical drapes, also termed surgical drapes, are commonly used in medical procedures to isolate a surgery site of a patient for treatment. These drapes are formed of sterile fabrics or fabric-like materials and protect the surgery site from contamination. In typical configurations, drapes define an opening that is placed at the surgery site using an adhesive or other sealing mechanism that functions to hold the drape in place, while the drape barrier extends outwardly from that opening providing a protective covering. As such, drape material and sizing are chosen to protect the subject and the surgery professionals from blood and other liquids, from dirt and other environmental contaminants, as well as reduce the incidence of surgical infection.

Drapes are routinely used in sterilized environments, such as emergency rooms, but drapes are also used in-theater for urgent response in environments that are often unpredictable and lacking suitable sterility to otherwise protect patients and surgeons. Furthermore, these environments often require immediate action by a tending physician or other health care provider, where patient outcomes can be affected by delays of even a few minutes. In such environments, a drape must be positioned quickly and in a manner that the drape does not interfere with the surgery about to take place.

There is, as a result, a need for better, more useful drape designs, designs that will increase surgical efficiency, reduce surgical error, and ideally improve patient outcomes and prospects.

### SUMMARY OF THE INVENTION

The present invention provides medical drapes that may be quickly deployed in place for protecting an area of a patient in need of a medical procedure. More specifically, the medical drapes include integrated toolkits that includes medical/surgical tools for use in the medical/surgical procedure. Furthermore, the medical drapes can also include selectively deployable adhesives for rapid fixturing of the drapes to the patient. The medical drapes are formed of fully or partially sterile materials and formed into packages having a stored state, in which the medical drape is itself protected against contamination, and a deployed state, in which the medical drape is placed on the patient and ready for the medical procedure. In the stored state, the medical drape houses or otherwise maintains the toolkit, such that the toolkit is also protected against contamination. Upon deployment of the medical drape, the toolkit is exposed for access by medical professionals, thereby allowing the toolkit to be used for the medical procedure.

In some examples, the tools are maintained in a toolkit housing that is adhesively attached to a fabric or fabric-like cover of the drape in the stored positioned, such that upon changing the drape to the deployed positioned the toolkit is positioned at or adjacent to a drape opening for ease of access near the site of the procedure. In some examples, an absolute position of the toolkit changes by the act of deploying the drape. For example, unpacking the drape cover can result in a change in the position of the toolkit relative to some fixed point on that cover. In some examples, the toolkit position does not change, just the state of the drape changes from stored to deployed to allow access to the toolkit.

In some examples, the toolkit itself has two states, a stored state that corresponds to the stored state of the drape, in which the toolkit is not accessible, but rather is preferably maintained sealed and sterile against contamination. In some examples, the toolkit has an exposed state that may or may not correspond to the deployed state of the drape. In some examples, when the drape is in the deployed state, the toolkit is still in the stored state, in that the toolkit tools are not accessible by a medical professional unless further actions are taken, such as removal of a toolkit cover layer. In yet other examples, the toolkit maybe placed in an exposed state, ready for access by medical professionals, when the drape is in the deployed state. In these later examples, the medical professional may find some or all of the tools in the toolkit immediately accessible, without further action, by virtue of the deployment of the drape. The integration of the toolkit with the drape cover allows for such varied configurations.

In some examples, the toolkit is integrated with the drape cover using an adhesive attachment mechanism or means. The attachment mechanism or means may be a removable attachment or a fixed attachment. In yet other examples, the attachment allows for adjustment of the toolkit relative to the drape cover. In some examples, the toolkit is integrated with the drape cover by or through a non-removable attachment. In some examples, the toolkit is integrated by forming some or all of a toolkit housing with the drape cover. For example, the drape cover can be integrally formed as the backing of the toolkit, which reduces the numbers of materials used and can make for a smaller-profile medical drape.

The toolkit may be formed to include one or more tools. The toolkits may include tools selected for specific medical procedures. In some examples the toolkits and their contents are: cricothyrotomy toolkit containing: (scalpel, hemostats, gauze, tracheal hook, syringes, local anesthetic, hypodermic needles, bougie, securing ties, antiseptic, etc.), central venous catheter insertion tool kits containing (scalpels, antiseptic, sutures, gauze, needle driver, syringes, hypodermic needles, scissors, occlusive dressings, guidewires, vessel dilators, central line, etc.), arterial catheter insertion toolkits containing (scalpel, antiseptics, gauze, local anesthetic, arterial catheter, guidewires, needed driver, suture, occlusive dressing, etc.) , tube thoracostomy insertion toolkits containing (scalpels, antiseptic, gauze, Kelley forceps, Rochester Pean forceps, sutures, needle driver, Heimlich valve, chest tube, syringes, hypodermic needles, sutures, occlusive dressings, etc.), urinary catheter insertion toolkits containing (antiseptic, gauze, lubrication, urinary catheter, collection back, syringes, etc.) laceration repair toolkits containing (sutures, needle driver, forceps, hemostats, scissors, hypodermic needs, syringes, local anesthetic, gauze, antiseptic, etc.), peripheral intravenous catheter insertion toolkits containing (intravenous catheters, antiseptic, tourniquet, gauze, syringes, adhesive tape, etc.).

In some examples, the tools within the toolkit are positioned for preferred access by a medical professional when the drape is in the deployed mode. That is, in some examples, the toolkit is positioned relative to the drape opening and the tools are arranged in the toolkit so that the medical professional accessing the tools does not have to turn or pivot to the toolkit (or tool holder), for access. Having such optimized tool orientation in an integrated toolkit decreases the chance of contamination during the medical procedure. And in emergency applications, the optimized tool orientation can help speed up execution of medical procedure steps. The tools, for example, may be placed in the toolkit in a particular order, such as the order of use during a procedure, and in a particular orientation, such as the orientation for which the medical professional would seek to access the tool for immediate use, without re-orienting the tool.

In some examples, individual tools are integrally attached to the drape cover, such that by moving the drape from the stored mode to the deployed mode, exposes one or more individual tools for immediate access by a medical professional. In some examples, some tools are in an exposed mode during the deployed mode, and thus immediately accessible to a medical professional, while other tools are maintained sealed, at least initially, while the drape is changed to the deployed mode.

In some examples, the drape can be adhesively attached to the patient. In some examples, the drape adhesive can be selectively deployed while the drape is positioned on the patient. In some examples, the drape adhesive can be selectively deployed via a pull tab(s) or other means such that the drape can remain substantially against the patient during adhesive activation.

In accordance with an example, a medical drape for placement on a subject for affecting a medical procedure, the medical drape comprises: a foldable drape cover for covering at least a portion of subject when the medical drape is in the deployed position, the foldable drape cover having an opening or access region configured to be place a target area of the subject in the deployed position, the foldable drape cover further having one or more adhesive attachment regions positioned to attach the foldable drape cover to the subject when the medical drape is in the deployed position, the foldable drape cover further configured to fold onto itself in a stored position of the medical drape; and a medical toolkit integrated to the foldable drape cover and configured to be protected from access when the foldable drape cover is in the stored position and configured to expose one or more tools for use in the medical procedure, when the medical drape is in the deployed position.

In accordance with an example, a drape is provided that includes a foldable drape cover for covering at least a portion of subject when the medical drape is in the deployed position, the foldable drape cover having an opening or access region configured to be place a target area of the subject in the deployed position, the foldable drape cover further having one or more releasable attachment regions positioned to attach the foldable drape cover to the subject when the medical drape is in the deployed position, the foldable drape cover further configured to fold onto itself in a stored position of the medical drape. The releasable attachment regions may be adhesive regions, for example.

In accordance with an example, a medical toolkit is provided with selective deployable releasable attachment regions, such as deployable adhesive regions, for allowing the toolkit to be attached to a drape, for example, at specific locations on the drape for use of the toolkit during a medical procedure.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a top view of an example central venous catheter insertion toolkit that may be integrated into a medical drape, in accordance with the teachings herein.

FIG. 2 is a top view of an example medial drape in a deployed position exposing a toolkit attached to the medial drape through an adhesive, in accordance with an example.

FIG. 3 is a perspective view of example tools of a cricothyrotomy toolkit that may be positioned in respective molded recesses of a toolkit, in accordance with an example.

FIG. 4 is a top view of a medical drape having an integrated cricothyrotomy toolkit, with tools like of the FIG. 3, in accordance with an example.

FIG. 5 illustrates an example deployed position of a medical drape with integrated toolkit and with an opening for a urinary catheter, in accordance with an example.

FIG. 6 is top view of an example chest tube toolkit that may be integrated with a medical drape, in accordance with an example.

FIG. 7 illustrates an example medial drape having an integrated chest tube toolkit and further including a collection chamber, a water seal chamber, and a suction chamber, with an opening allowing a chest tube to extend into/from the patient through an opening, in accordance with an example.

FIG. 8A illustrates a medical drape, with integrated toolkit, in a stored state, in accordance with an example. FIG. 8B illustrates an upper side of the medical drape of FIG. 8A in a deployed state, in accordance with an example. FIG. 8C illustrates an underside of the medical drape of FIG. 8A in a deployed state, in accordance with an example.

FIG. 9A-9C illustrate a top view, an isometric view, and an underside view, respectively, of a medical drape having a pull tab to expose an adhesive backing, in accordance with an example.

### DETAILED DESCRIPTION

The present invention provides medical drapes that may be quickly deployed in place for protecting an area of a patient in need of a medical procedure. More specifically, the medical drapes include integrated toolkits that includes medical/surgical tools for use in the medical/surgical procedure. The medical drapes are formed of fully or partially sterile materials and formed into packages having a stored state, in which the medical drape is itself protected against contamination, and a deployed state, in which the medical drape is placed on the patient and ready for the medical procedure. In the stored state, the medical drape houses or otherwise maintains the toolkit, such that the toolkit is also protected against contamination. Upon deployment of the medical drape, the toolkit is exposed for access by medical professionals, thereby allowing the toolkit to be used for the medical procedure.

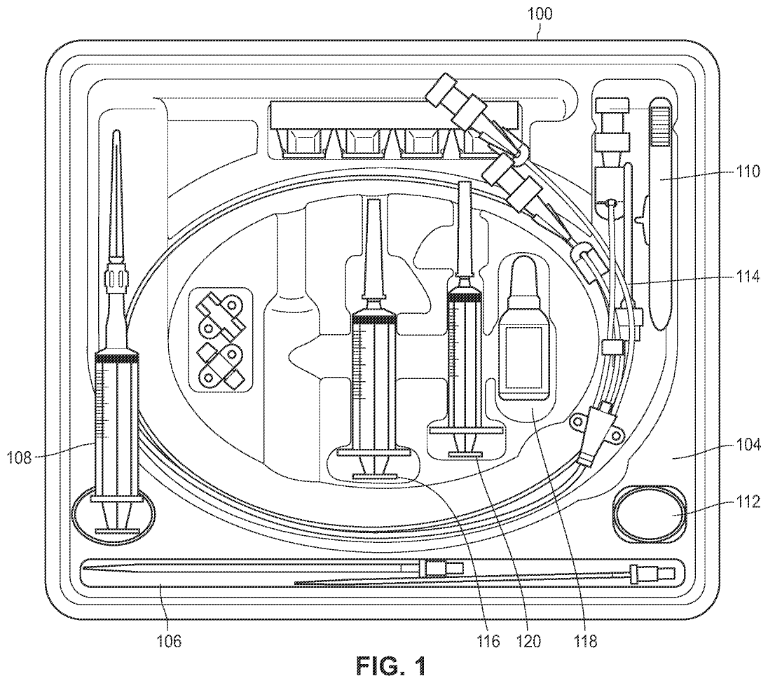

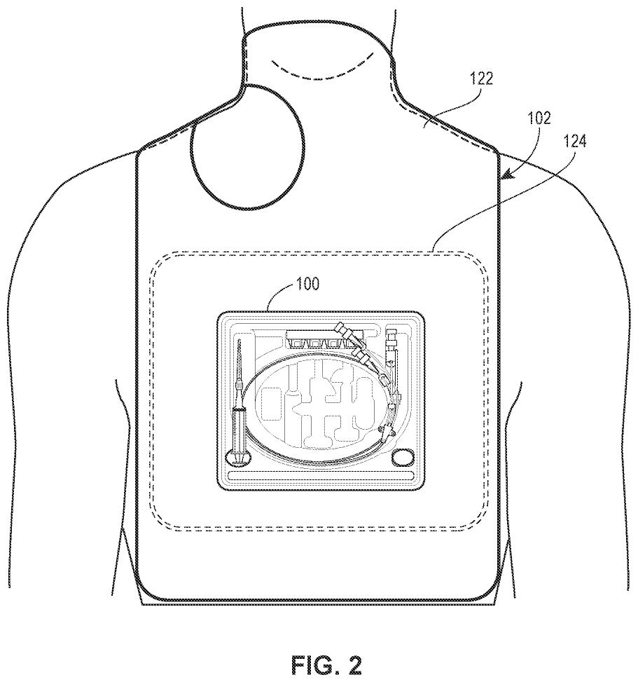

FIG. 1 illustrates an example central venous catheter insertion toolkit 100 as may be integrated into a medical drape 102, as shown in FIG. 2. The central line toolkit 100 has a receptacle tray 104 that functions as a housing with molded recesses for holding a plurality of different tools, including a dilator 106, a syringe and needle 108, a scalpel 110, a sponge 112, guide syringe and needle 114, anesthesia syringe and needle 116, lidocaine bottle 118 with syringe and needle 120. As with other toolkits described herein, in some examples, the tray (or housing) 104 maintains the tools against free release regardless of the orientation of the toolkit 100. For example, FIG. 2 shows the 100 in the horizontal plane configuration, which the tools are maintained against free release, until a medical professional selects the tool and releasably removes the tool from its recess in the toolkit 100, while leaving the other tools in their respective recesses. If the toolkit 100 were rotated 90° into vertical plane orientation, the housing 104 would still maintain the tools against free release. Free release refers to unintended release of the tool without a medical professional or other personnel actively removing the tool, for example, by removing the tool from a press fitting within a respective recess.

The toolkit 100 is integrated into the medical drape 102, which further includes a drape cover 122 for protecting the patient. The drape 102 is shown in a deployed position in FIG. 2, where an adhesive backing 124 is placed on the drape cover 112 and coinciding with the position of the toolkit 100, such that the drape cover 122 may be held in place on the subject by adhesion, thereby maintaining a steady position of the toolkit during a medical procedure. The adhesive on the drape cover can also be selectively exposed/activated. For example, in the stored position the adhesive can be covered by a protective layer, such as a thin film. This thin film can then be peeled away to expose the adhesive so it can be securely attached to the patient. Said thin film could be exposed either before placing the drape on the patient or after placing the drape on the patient. To aid in exposing the adhesive after placing the drape on the patient, one or more pull tabs can be added to the protective adhesive layer. These one or more pull tabs can be arranged such that when the drape is placed on the patient the pull tab(s) are easily accessible by the clinician. For example, the pull tab could extend beyond the side of the drape for ease of access. The pull tab could also extend through a slit or opening in the drape. Furthermore, the pull tab can be attached to the drape. This attachment can be designed such that when sufficient force is applied to the pull tab, it is detached from the drape and is able to peel off the adhesive protective layer. Such an arrangement can allow the medial professional to perform a medical procedure without having to turn or pivot unnecessarily during use. All tools are in the direct line of sight of the medical professional. The tray 104 is orientated for optimum use by the medical professional and the recesses in the tray 104 are optimally positioned for access by the respective tool. This will decrease the chance of contaminating the procedure site or loosing tools, and in cases of emergencies will help speed the execution of the procedure. Such configurations can be particularly useful when procedures must be performed during medical transport such as in medical aircraft or ground ambulances. Each of the tools can be a sterile or non-sterile. The toolkit 100 may include a cover that is removable when the drape 102 is in the deployed mode to expose one or more of the tools for operation.

In some examples, the toolkit 100 is removable from the drape cover 122, for example, through a releasable mechanism such as an adhesive layer, hook and loop fastener(s), clips, or slots between a backing of the toolkit and an upper surface of the drape cover.

Any number of toolkits may be used in place of the toolkit 100.

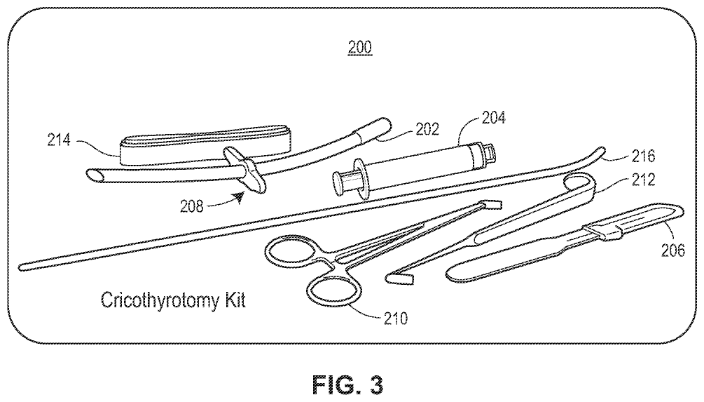

FIG. 3 illustrates tools of cricothyrotomy toolkit 200 that may be configured into a tray of molded recesses for each tool. The kit 200 includes an endotracheal tube 202, which may be cuffed for maintaining an open airway, a syringe 204, a sterile scalpel blade 206 for incisions, a syringe connector/adapter 208 for inflation of airway cuff, curved Kelly forceps 210 for clamping or opening, a tracheal hook 212, cotton tape 214, and a bougie 216.

In examples herein, toolkits are formed with housings that include recesses for maintaining tools in place. In some examples, the housing has a separate recess for each tool. The housing and recesses may be formed of a pliable polyurethane or other biocompatible plastic or paper material. In some examples, the recesses are configured into press-fits for maintaining tools in place, through a pinching fit, until removed by a medical professional. In some examples, the recesses have flanged openings for maintaining tools in place, until removed by a medical professional. In some examples, the housing is configured to maintain the tools against release at different orientations of the toolkit during a medical procedure. For example, the housing may be configured, through flanged openings on recesses, press-fits, graduated weighting changes over the housing, or other features to maintain tools in place whether the toolkit is orientated in a horizontal plane, in a vertical plane, or tilted between those two orientations.

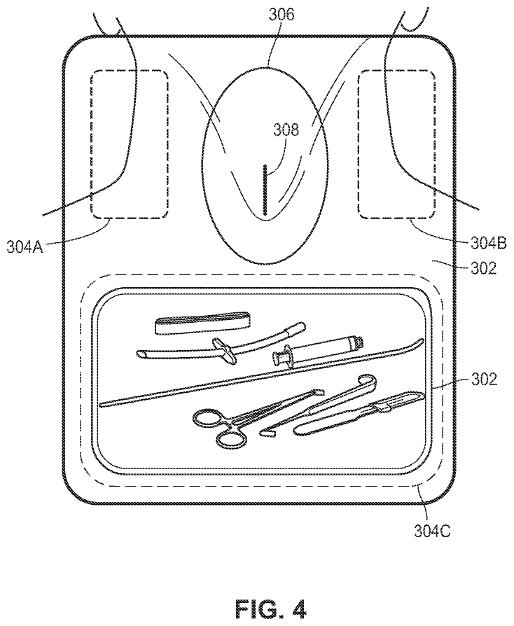

FIG. 4 illustrates a medical drape 300 having an integrated cricothyrotomy toolkit 302. The drape 300 includes adhesive backings 304 and three different locations for maintaining the drape in place during a procedure when the drape 300 is deployed. Two backings 304A and 304B are positioned adjacent to and on opposing sides of a drape access panel 306 that defines a region over which a medical professional can access the patient below the drape. The access panel may be formed of the same material as the drape 300 and may include a preformed slot 308 for access. In some examples, the preformed slot 308 defines an incision location.

Any number of toolkits may be integrated into a drape. These include, without limitation, cricothyrotomy kits, tube thoracostomy kits, central venous catheter placement kits, arterial catheter placement kits, urinary catheter placement kits, suture kits, and peripheral intravenous catheter placement and laceration repair kits.

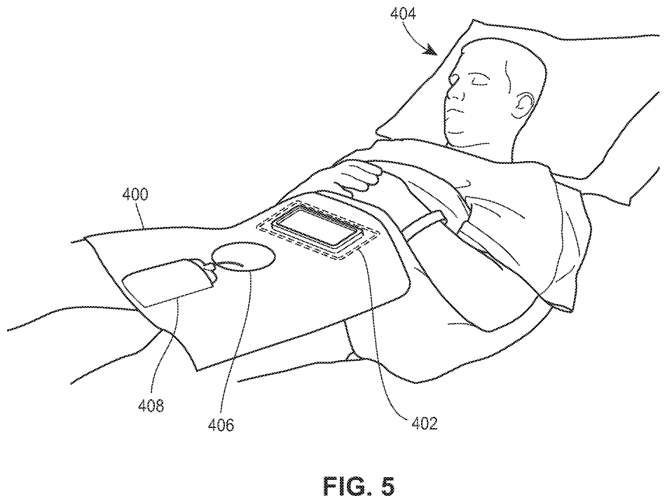

FIG. 5 illustrates an example deployed position of a medical drape 400 with integrated toolkit 402 for insertion of a urinary catheter. The drape 400 is positioned on a subject 404 and maintained in place by an adhesive backing, not shown. The drape 400 is configured such that in the stored state, the adhesive is not exposed for attachment to the subject 404. Instead, by opening the drape 400, the adhesive is exposed. In some examples, the adhesive is exposed only as the drape 400 is been fully opened to cover the subject matter, for example, the drape 400 is in a multi-fold configuration in the stored state. When deployed, the drape 400 exposes an opening 406 for access by medical professions. In the illustrated example, the drape 400 further includes a catheter pouch bag 408 that is also exposed for use in the deployed state. The integrated toolkit 402 can be any type of toolkit, depending on the application of use. Further, in some examples, the toolkit may be swapped for another toolkit, only after the drape has been deployed, for example, by a releasable mechanism at the interface between the toolkit and the drape cover.

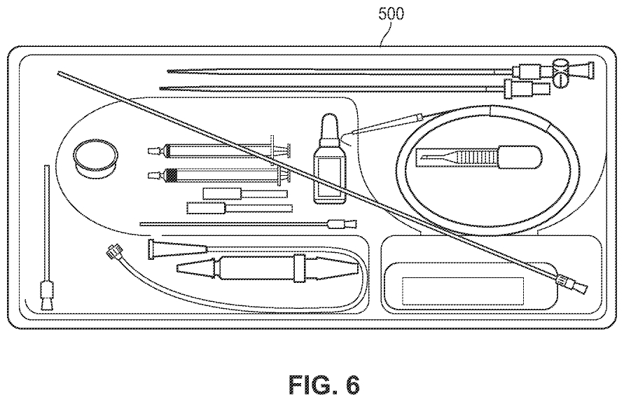

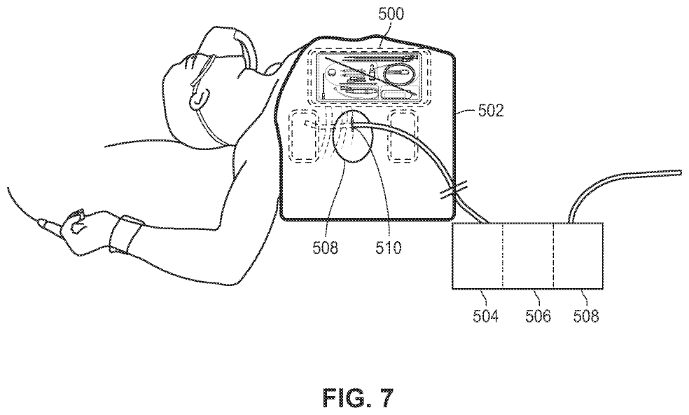

FIG. 6 illustrates another example toolkit in the form of a chest tube toolkit 500. FIG. 7 illustrates a drape 502 with the chest tube toolkit 500 integrated therewith and further including a collection chamber 504, a water seal chamber 506, and a suction chamber 506, with an opening 508 allowing a chest tube to extend into/from the patient through an insertion location 510 in the opening 508. Adhesive regions are shown in dashed lines.

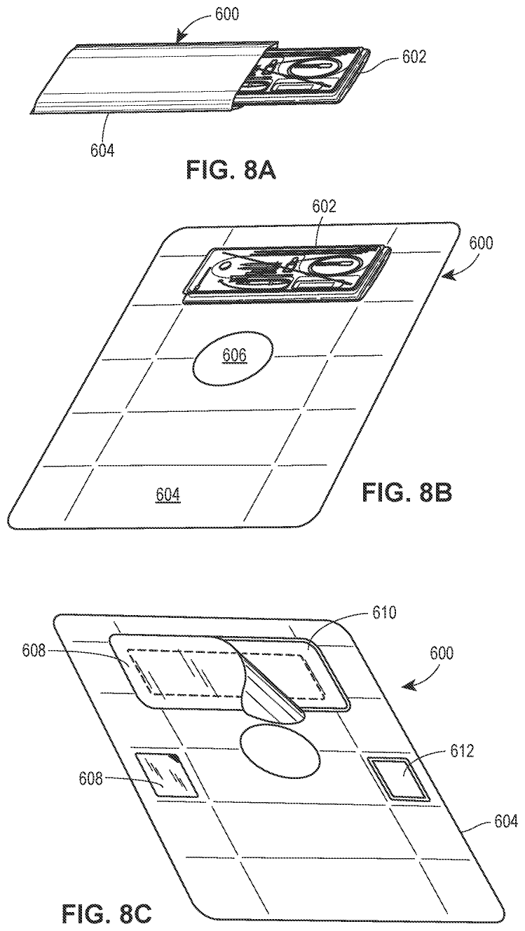

FIGS. 8A and 8B illustrate a drape 600 with integrated toolkit 602 in a stored stated (FIG. 8A) and a deployed state (FIG. 8B), respectively. The drape 600 is formed with a drape cover 604 that is folded in the stored state and completely or partially unfolded in the deployed state, exposing an opening 606 in the later. FIG. 8C illustrates an underside of the drape 600, showing adhesive release patches 608 that serve as a protective covering but that are removable to expose the adhesive patches underneath for attaching to a subject. A top adhesive release patch 608 may be positioned under the toolkit 602 and sized to be co-extensive with the undersurface of thereof or sized over a portion sufficient to maintain the toolkit region of the drape 600 in a relatively fixed positioned when the patch 608 is removed (shown partially removed) and an adhesive layer 610 under near is exposed. Thus, in some examples, the toolkit 602 is adhesively attached to the top surface of the drape 600 and another adhesive 610 may be positioned on a lower surface of the drape 600. The toolkit 602 may be removed and repositioned on the drape 600 via the adhesive engagement between the two. While the adhesive layer 610 may be exposed to retain the drape in place in a region that coincides with that of the toolkit. Of course, in some examples, the toolkit may be affixedly integrated in the drape. Another release patch 608 is shown in the middle left and shows a corner slightly pulled back to show partially show an adhesive layer underneath. In the middle right, the release patch has been removed and leaving exposed an adhesive layer 612 that, combined with the adhesive layer 610 and any other adhesive layers act as attachment regions positioned to attach the medical drape 600 to the subject when the medical drape 600 is in the deployed position.

The adhesion between the toolkit and drape cover may be achieved in numerous different ways and is not limited to use of an adhesive material. Furthermore, the releasable attachment between the toolkit and the drape cover can be configured to allow the toolkit to be moved into place and adhere to the drape cover at specified locations and in specific or various orientations selected by the medical professional during placement. In some examples, the toolkit and the drape cover are releasably attached through a VELCRO engagement between marrying patches on the toolkit and the drape cover. In some examples, the releasable attachment, e.g., the adhesive, may be activatable. For example, a unifying pull tab may extend outwardly from the toolkit for access by medical personal and configured to expose one or more adhesive patches. For example, to activate the adhesive, a medical professional could hold the drape with one hand over the desired spot to be placed, then with the other hand pull the pull tab which then exposes the adhesive instantly sticking it to where you have held the drape. The pull tab may he placed such that is easy to grab with the toolkit positioned in place. For example, the pull tab may extend out from the side of the toolkit. There may be multiple discrete adhesive zones all attached together by the pull strip, or it could be one single adhesive zone.

In examples, where the toolkit is pre-attached to the drape cover during deployment and is to be released when the drape is deployed for a medical procedure, the toolkit may be releasably be attached to the drape cover through one or more adhesive patches, VELCRO attachments, perforated film having a pull release, a pull tab mechanism, a zipper, or any combination thereof, or any other suitable attachment mechanism, each of which may be considered attachment means herein.

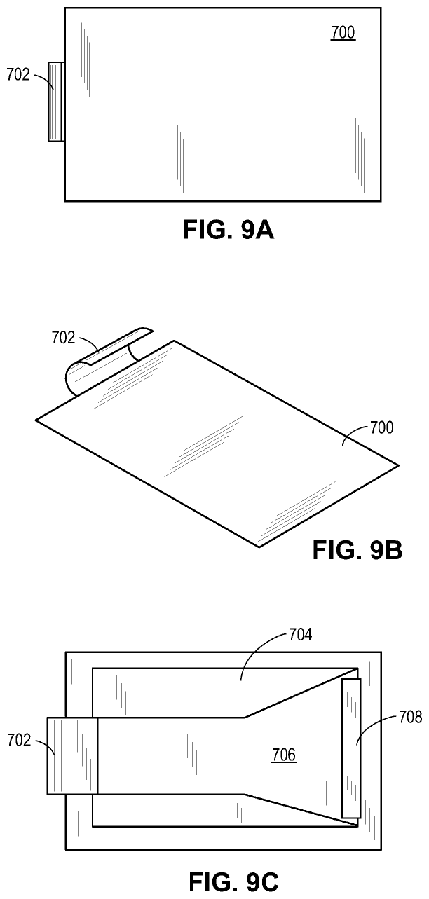

FIGS. 9A, 9B, and 9C illustrate top view, isometric view, and underside view, respectively, of a drape 700 having a pull tab 702 to expose an adhesive backing 704. The adhesive configuration of the drape 700 can be used to attach the toolkit to the person, the toolkit to the drape, or the drape to the person, in different embodiments.

The pull tab 702 may be accessible when the drape or toolkit is already positioned flush against the object you want it to stick to. The pull tab 702 can be accessible from the side of the drape/toolkit or also through a slot through the drape/toolkit. In some examples, the releasable attachment, e.g., a cover 706 for the adhesive backing 704, may be activatable. For example, a unifying pull tab 702 may extend outwardly from the toolkit for access by medical personal and configured to expose one or more adhesive patches. For example, to activate the adhesive, a medical professional could hold the drape 700 with one hand over the desired spot to be placed, then with the other hand pull the pull tab which then exposes the adhesive instantly sticking it to where you have held the drape 700. The pull tab 702 may be placed such that is easy to grab with the toolkit positioned in place. For example, the pull tab 702 may extend out from the side of the toolkit. There may be multiple discrete adhesive zones all attached together by the pull strip, or it could be one single adhesive zone.

In some examples, the pull tab 702 is preferably attached to the adhesive strip cover at a location 708, and the rest of the pull tab 702 is not attached. In some examples, the pull tab 702 is preferably is connected to the opposing edge of the adhesive backing 704. That way, when the pull tab is pulled by the user, the pull tab will tend to strategically peel the adhesive backing off starting at the opposite edge. The user can then continue to pull to reveal the full adhesive strip or strips (e.g., if multiple adhesive backings were attached to a single pull tab).

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A medical drape for placement on a subject for affecting a medical procedure, the medical drape comprising:
a foldable drape cover for covering at least a portion of subject when the medical drape is in the deployed position, the foldable drape cover having an opening or access region configured to be placed at a target area of the subject in the deployed position,
the foldable drape cover further having one or more releasable attachment regions positioned to attach the foldable drape cover to the subject when the medical drape is in the deployed position,
the foldable drape cover further configured to fold onto itself in a stored position of the medical drape; and
a medical toolkit integrated into the foldable drape cover and configured to be protected from access when the foldable drape cover is in the stored position and configured to expose one or more tools for use in the medical procedure, when the medical drape is in the deployed position.

2. The medical drape of claim 1, wherein the medical toolkit is adhesively attached to the foldable drape cover as the releasable attachment regions, and wherein the medical toolkit is configured to be removed from integration to the foldable drape cover.

3. The medical drape of claim 1, wherein the medical toolkit is affixedly integrated into the foldable drape cover to prevent movement of the medical toolkit in the deployed position.

4. The medical drape of claim 1, wherein the medical toolkit is releasably attached to the foldable drape cover by an attachment selected from the group consisting of adhesive patches, VELCRO material, a pull tab, a zipper, a perforated film, and any combination thereof.

5. The medical drape of claim 1, wherein the one or more releasable attachment regions are positioned on an undersurface of the foldable drape cover and each releasable attachment region comprises an adhesive material and a protective covering that is exposed for removal when the medical drape is in the deployed position.

6. The medical drape of claim 1, wherein the one or more releasable attachment regions are positioned on an undersurface of the foldable drape cover and each releasable attachment region comprises an adhesive material and a protective covering that is removed when the medical drape is transitioned from the stored position to the deployed position.

7. The medical drape of claim 1, wherein the medical toolkit has a removable cover.

8. The medical drape of claim 7, wherein the movable cover is automatically removed when the medical drape is transitioned from the stored position to the deployed position.

9. The medical drape of claim 1, wherein the medical toolkit comprises a housing with recesses for each tool in the toolkit.

10. The medical drape of claim 9, wherein the recesses of the housing comprise a separate recess for each tool, and wherein each recess has an opening shaped to a corresponding tool.

11. The medical drape of claim 9, wherein at least some of the recesses comprise flange opening for maintaining respective tool with the drape with the toolkit in different orientations.

12. The medical drape of claim 9, wherein at least some of the recesses are press-fit recess for respective tool.

13. The medical drape of claim 1, wherein the medical toolkit has a housing for housing tools of the toolkit, wherein the housing is configured to maintain the tools against free release at different orientations of the toolkit during a medical procedure.

14. The medical drape of claim 13, wherein different orientations comprise an in-horizontal plane orientation and an in-vertical plane orientation.
